(19) United States
(12) Patent Application Publication
(10) Pub. No.: US 2019/0246931 A1
(43) Pub. Date: Aug. 15, 2019

(54) IMPLANTABLE MULTIELECTRODE ARRAY, METHOD FOR PRODUCING AN IMPLANTABLE MULTIELECTRODE ARRAY AND DEVICE FOR PERFORMING THE METHOD

(71) Applicant: DYCONEX AG, Bassersdorf (CH)

(72) Inventors: ECKARDT BIHLER, WINTERTHUR (CH); HUBERT ZIMMERMANN, STAUFEN (CH)

(21) Appl. No.: 16/249,967

(22) Filed: Jan. 17, 2019

(30) Foreign Application Priority Data
Feb. 14, 2018 (EP) 18156702.5-1124

(51) Int. Cl. A61B 5/0478; H01R 43/16; A61N 1/05

(52) U.S. Cl. CPC A61B 5/0478; A61B 2562/125; A61N 1/0551; H01R 43/16

(57) ABSTRACT

A method for producing an implantable multielectrode array includes providing a substrate carrying conductors each having a section with a constriction. The sections extend mutually parallel in a first direction. A portion of the substrate is removed to form separate first and second substrate parts separated by a gap. Each section extends in the first direction from the first substrate part across the gap to the second substrate part. A first force is exerted on the first substrate part, a second force is exerted on the second substrate part and the sections are heated to generate a fracture of the sections at the constriction. The fracture separates the section into an electrode protruding from the first substrate part and an electrode protruding from the second substrate part. An implantable multielectrode array and a device for manufacturing an implantable multielectrode array are also provided.

### CROSS-REFERENCE TO RELATED APPLICATION

This application claims the priority, under 35 U.S.C. § 119, of European Patent Application EP 1 815 6702.5-1124, filed Feb. 14, 2018; the prior application is herewith incorporated by reference in its entirety.

### BACKGROUND OF THE INVENTION

### Field of the Invention

The present invention relates to a method for producing an implantable multielectrode array, an implantable multielectrode array, as well as a device for producing an implantable multielectrode array.

Such arrays are known in the prior art. For instance, U.S. Pat. No. 5,215,088 A discloses a multielectrode array including electrodes formed of silicon. Furthermore, European Patent EP 2 185 236 B1, corresponding to U.S. Pat. Nos. 8,209,023; 8,489,193; 9,302,107; 9,592,377; and 10,052,478, discloses a multielectrode array including spike electrodes made from metals such as e.g. platinum, iridium, and alloys of platinum and iridium.

Concerning manufacturing of multielectrode arrays of the afore-mentioned kind it has proven generally difficult to manufacture such arrays in a simple and efficient manner. In European Patent EP 2 185 236 B1, corresponding to U.S. Pat. Nos. 8,209,023; 8,489,193; 9,302,107; 9,592,377; and 10,052,478, for example, the spike electrodes are separately produced and then affixed onto the substrate of the multielectrode array.

### SUMMARY OF THE INVENTION

Therefore, it is an objective to provide a method for producing an implantable multielectrode array, an implantable multielectrode array, as well as a device for producing an implantable multielectrode array which are improved in this regard.

With the foregoing and other objects in view there is provided, in accordance accordance with the invention, a method for producing an implantable multielectrode array, which comprises the steps of:
a) providing a substrate on which a plurality of conductors are disposed, wherein each conductor includes a section including a constriction, and wherein the sections extend parallel to one another in a first direction,
b) removing a portion of the substrate such that a first and a separate second substrate part is formed, which substrate parts are separated by an air gap, and wherein each section extends in the first direction from the first substrate part across the gap to the second substrate part, and
c) exerting a first force on the first substrate part and a second force on the second substrate part and heating the sections, such that a fracture of the respective section is generated at the respective constriction, which fracture separates the respective section into an electrode protruding from the first substrate part and an electrode protruding from the second substrate part.

The result of this method is a first multielectrode array made of the first substrate part with a plurality of tips (electrodes) protruding from its surface and a potential second multielectrode array made of the second substrate part with a plurality of tips (electrodes) protruding from its surface.

Particularly, the present invention allows the production of permanently implantable, and particularly biostable multielectrode arrays, particularly for measuring brain waves, in an efficient manner.

Particularly, due to the principle according to which the respective electrode tip is manufactured, the present invention allows the use of exclusively biocompatible/biostable materials regarding the substrate and the electrodes, such as polymers and metals (see also below).

Furthermore, the method constitutes an efficient process that can be based on printed circuit board techniques and can be used to produce implantable multielectrode arrays in large quantities.

Due to the construction of the implantable multielectrode array, comparatively low production costs can be achieved and particularly the exclusive use of biostable materials is possible.

Furthermore, particularly, the electrodes protruding from the first substrate part and/or the electrodes protruding from the second substrate part can be used as electrodes of the multielectrode array to be produced. Particularly, the multielectrode array can also be a one-dimensional array, in which case each of the substrate parts with the respective electrodes (or only one of the two substrate parts) can form a multielectrode array. Of course, the respective electrodes can be subject to further processes such as cleaning and/or coating with other materials (see e.g. below).

Furthermore, according to an embodiment of the method, the second force points in the first direction and the first force points in a second direction that is opposite the first direction, and wherein particularly the forces are of equal magnitude. This allows pulling the substrate parts and the portions of the conductors on either side of the respective constriction apart in a defined manner.

Furthermore, according to an embodiment of the method, the respective fracture is a ductile fracture. Thus, particularly, the heated sections of the conductors are torn apart at the respective constriction by the opposite forces such that the respective fracture separating the respective section into the respective two electrodes is a ductile fracture. This particularly allows generating electrodes that continuously taper towards the tip of the respective electrode.

Furthermore, according to an embodiment of the method, a plurality of first and second substrate parts are generated by repeating the steps a) to c).

Furthermore, according to an embodiment of the method, for forming the multielectrode array, a plurality of substrate parts (including first and/or second substrate parts) is bonded together to form a substrate of the implantable multielectrode array so that the electrodes protrude from a surface of the substrate of the multielectrode array formed by the plurality of substrate parts bonded together. This can be achieved for example by stacking a plurality of one dimensional multielectrode arrays produced with the method of the present invention on top of each other and by joining them, for example with a low melting LCP. This process allows at the same time hermetically sealing the conductors. The number of one dimensional multielectrode arrays to be stacked on top of each other depends on the number of electrodes and the size of the two dimensional multielectrode array that is to be obtained. The distance between the electrode layers is preferably between 0.02 mm to 0.1 mm. Also here, particularly, each fracture of the conductor sections preferably is a ductile fracture.

In one embodiment, the plurality of conductors with sections disposed in parallel is formed of preferably of at least 2, preferably at least 3, even more preferably at least 5 and most preferably at least 10 parallel conductors. This results in one dimensional multielectrode arrays that respectively include at least 2, 3, 5 or 10 parallel electrodes. The two dimensional microelectrode preferably includes at least 2, preferably at least 3, even more preferably at least 5 and most preferably at least 10 one dimensional electrode microarrays stacked on top of each other.

Furthermore, according to an embodiment of the method, the section of the respective conductor is heated to a temperature in the range from 100° C. to 200° C. for generating the (ductile) fractures under tension.

Furthermore, according to an embodiment of the method, the gap formed in the respective initial substrate extends in a third direction that is perpendicular to the first and/or second direction.

Furthermore, according to an embodiment of the method, each of the sections of the conductors includes a center axis, extending in the first direction, wherein the center axes are equidistantly spaced in the third direction, and wherein a distance between each two neighboring sections (or portions) in the third direction lies within the range from 0.05 mm to 1 mm (grid dimension). This grid dimension corresponds to the density of neurons in the brain. Each of the electrodes is therefore able to, on average, contact one neuron when implanted.

Furthermore, according to an embodiment of the method, the constrictions can be aligned with respect to the third direction. However, alternatively, the constrictions can also be aligned with a pre-defined curved line so that the tips of the respective electrode of a substrate part are located on the line (or on a defined curved surface when considering the whole array).

Furthermore, according to an embodiment of the method, the gap includes a width in the first direction that lies within the range from 1 mm to 5 mm.

Furthermore, according to an embodiment of the method, the respective conductor disposed on the substrate includes a width (e.g. along a surface of the substrate and particularly perpendicular to the first direction) outside the respective constriction that lies in the range from 20 μm to 200 μm.

Furthermore, according to an embodiment of the method, the conductors disposed on the support (e.g. before forming the gap) include a thickness (e.g. perpendicular to the width and/or normal to the surface of the substrate) that lies in the range from 10 μm to 50 μm.

Furthermore, according to an embodiment of the method, the respective constriction includes a length in the first direction that is within the range from 50 μm to 200 μm.

Furthermore, according to an embodiment of the method, a smallest width of the respective constriction (e.g. along a surface of the substrate and particularly perpendicular to the first direction) amounts to 20% to 80% of the width of the respective conductor outside the respective constriction (see above).

Furthermore, according to an embodiment of the method, for forming the gap, the material of the substrate is removed by one of: laser ablation, plasma etching or chemical etching. Other methods are also conceivable.

Furthermore, according to an embodiment of the method, the conductors are formed on the substrate by coating the substrate with a conductive material, particularly with a metal, preferably gold (Au).

Furthermore, according to an embodiment of the method, the substrate includes or is formed of a thermoplastic polymer, particularly a liquid crystal polymer, which is particularly biocompatible and/or biostable.

Furthermore, according to an embodiment of the method, for forming the conductors on the substrate, the substrate (e.g. LCP, see above) can be coated with the conductive material (e.g. gold) using a galvanic process, wherein a layout of the conductors can be defined before using photolithography.

Furthermore, according to an embodiment of the method, the respective conductor is formed from a photolithographically defined conductor track applied to the substrate by galvanic reinforcement of the respective conductor track.

Furthermore, according to an embodiment of the method, each electrode is coated in a further step of the method with a conductive coating.

Particularly, according to an embodiment, the coating includes or is formed of platinum, iridium, or an alloy of platinum and iridium, or a similar conductive material. The advantage of such a coating, for example in the case of gold electrodes, is that the diffusion of gold when implanted in the brain is reduced.

Furthermore, according to an embodiment of the method, each electrode includes a tip that is coated with a conductive coating.

According to a further aspect, a device for performing the method according to the present invention for producing an implantable multielectrode array is disclosed. The device comprises:
a first substrate holder for holding the first substrate part,
a second substrate holder for holding the second substrate part,
a heater for heating the sections of the conductors, and
an actuator configured to move the two substrate holders apart (e.g. upon heating of the sections of the conductors that are to be separated by e.g. ductile fracture) for exerting the first force on the first substrate and the second force on the second substrate.

Particularly, in an embodiment of the device, the heater can be configured to produce heated air that is directed towards the sections by using a nozzle of the heater.

Furthermore, in an embodiment, the device can include a gear unit or leadscrew via which the two substrate holders are coupled, wherein the actuator (e.g. stepper motor) can be configured to act on the gear unit/lead screw in order to move the substrate holders apart (or closer to one another, particularly for holding the substrate parts when generating the gap).

Yet another aspect relates to an implantable multielectrode array produced by the method according to the present disclosure.

Furthermore, a further aspect relates to a multielectrode array, comprising a plurality of metallic conductors (e.g. wires) embedded in an insulating substrate such that an end section of each conductor protrudes from a surface of the substrate, wherein the respective end section forms an electrode including a drawn tip.

Furthermore, in an embodiment of the implantable multielectrode array, the respective electrode includes a fracture surface of a ductile fracture at the tip of the respective electrode.

Furthermore, according to an embodiment, the respective tip, particularly the fracture surface, is coated with an electrically conductive coating, wherein the coating particularly includes or is formed out of: platinum, iridium, or an alloy of platinum and iridium.

Furthermore, in an embodiment, the electrodes extend parallel to one another, particularly normal to the surface.

Furthermore, according to an embodiment of the implantable multielectrode array, the electrodes of the multielectrode array are disposed on a virtual grid on the surface of the substrate of the multielectrode array.

Particularly, the grid can be e.g. a two-dimensional square lattice having a grid dimension in the range from 20 μm to 0.5 mm. Here, the grid dimension is the distance between each two electrodes that are the nearest neighbors.

Furthermore, according to an embodiment of the implantable multielectrode array, the substrate is a thermoplastic polymer, particularly a liquid crystal polymer.

Furthermore, according to an embodiment of the implantable multielectrode array, the respective conductor has a diameter in the range from 10 μm to 100 μm.

Further, according to an embodiment of the implantable multielectrode array, the respective tip has a radius in the range from 0.2 μm to 5 μm.

Furthermore, according to an embodiment of the implantable multielectrode array, a length of the respective electrode over which the respective electrode protrudes with its tip past the surface of the substrate of the implantable multielectrode array lies in the range from 0.02 mm to 3 mm (e.g. in the range from 0.2 mm to 3 mm).

Furthermore, according to an embodiment of the implantable multielectrode array, a length of the respective electrode over which the respective electrode is tapered lies in the range from 10 μm to 3 mm.

Furthermore, according to an embodiment of the implantable multielectrode array, a region of the respective conductor that protrudes out of the substrate/surface is coated with a metal or an insulator, particularly one of silicon oxide, titanium, titanium oxide, or silicon nitride.

Furthermore, according to an embodiment of the implantable multielectrode array, the tips are disposed in a common plane or in a pre-defined curved surface in order to follow the course of an object in which the electrodes are to be inserted with their tips leading.

According to yet another embodiment of the implantable multielectrode array, the substrate has a curved shape.

Particularly, the substrate can include a first portion integrally connected to a second portion of the substrate, wherein the second portion extends at an angle with respect to the first portion. Particularly, the second portion can extend perpendicular to the first portion.

Particularly, according to an embodiment, the surface of the substrate from which the electrodes of the implantable multielectrode array protrude is formed by a face side of the second portion, so that particularly the electrodes extend parallel to one another at the angle (particularly perpendicular) with respect to the first portion of the substrate.

According to a further embodiment, the implantable multielectrode array includes a multiplexer chip embedded into the substrate for passing electrical signals to individual electrodes.

According to a further embodiment of the implantable multielectrode array, the implantable multielectrode array includes an electrical coil for receiving data and electrical energy transmitted to the implantable multielectrode array. Particularly, the coil is embedded in the substrate of the implantable multielectrode array.

Other features which are considered as characteristic for the invention are set forth in the appended claims.

Although the invention is illustrated and described herein as embodied in an implantable multielectrode array, a method for producing an implantable multielectrode array and a device for performing the method, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a diagrammatic plan view of a multielectrode array during manufacturing;

FIG. 2 is a diagrammatic plan view of a multielectrode array after having manufactured the electrodes of the multielectrode array;

FIG. 3 is a device for producing a multielectrode array;

FIG. 4 is an embodiment of a multielectrode array having a curved substrate;

FIG. 5 is a plan view of an embodiment of a multielectrode array having a multiplexer chip and a coil for data communication and for receiving electrical energy; and

FIG. 6 is a perspective view which shows an application of the multielectrode array of FIG. 4 to a peripheral nerve.

### DETAILED DESCRIPTION OF THE INVENTION

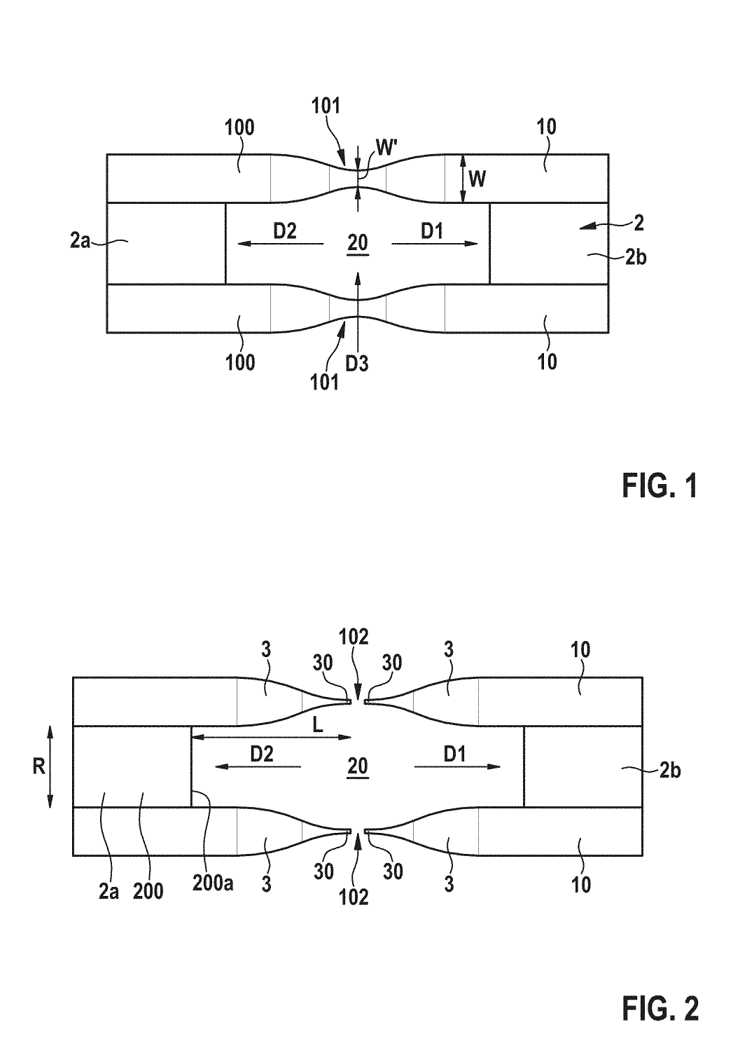

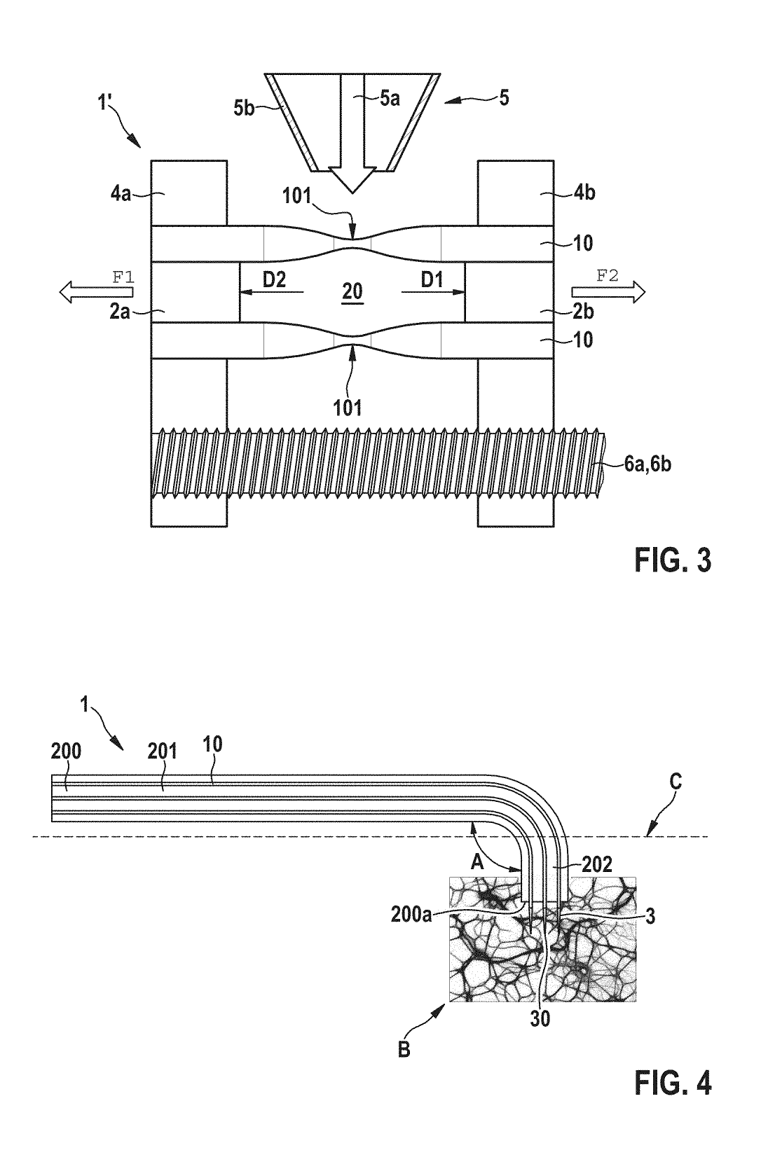

Referring now to the figures of the drawings in detail and first, particularly, to FIG. 1 in conjunction with FIGS. 2 and 3 thereof, there is seen an implantable multielectrode array 1 produced by a method according to the invention. According to an embodiment of the method, an insulating substrate 2 (e.g. LCP) is provided (cf. FIG. 1) on which a plurality of conductors 10 are disposed that are preferably made out of gold, wherein each conductor 10 includes a section 100 including a constriction 101, and wherein the sections 100 extend parallel to one another in a first direction D1.

The conductors 10 can be formed and may include the dimensions as described herein.

Further, a portion of the substrate 2 is removed such that a first and a separate second substrate part 2a, 2b are formed that are separated by an air gap 20, wherein each section 100 extends in the first direction D1 from the first substrate part 2a across the gap 20 to the second substrate part 2b.

Then, as indicated in FIG. 2, a first force F1 is exerted on the first substrate part 2a and a second force F2 is exerted on the second substrate part 2b and at the same time the sections 100 are heated, particularly to a temperature in the range from 100° C. to 200° C. Here, in order to pull the substrate parts 2a, 2b apart, the second force F2 is directed in the first direction D1 and the first force F1 (that is of equal magnitude) is directed in a second direction D2 opposite the first direction D1. In this fashion, ductile fractures 102 are preferably generated at the constrictions 101 that separate the respective section 100 into an electrode 3 protruding from the first substrate part 2a and an electrode 3 protruding from the second substrate part 2b.

As indicated in FIG. 2, such a substrate part 2a can form the final substrate 200 of the implantable multielectrode array 1 with electrodes 3 protruding from a surface 200a of the substrate 200 (e.g. in case of a one-dimensional array). However, in order to construct two-dimensional arrays, several such substrate parts 2a and/or 2b can be bonded together (stacked on top of each other) to form a substrate 200. Then, these substrate parts 2a and/or 2b bonded together jointly form the surface 200a from which the electrodes 3 that are now disposed on a 2D grid protrude. The grid dimension R (cf. FIG. 2) can have the values described herein.

FIG. 3 shows a device 1′ that can be used to conduct the process described with reference to FIGS. 1 and 2.

The device 1′ is particularly adapted for the controlled application of a tractive force (e.g. Forces F1 and F2) while simultaneously heating the sections 100 of the conductors 10 extending across the gap 20. The two substrate parts 2a, 2b are fixed on two substrate holders 4a, 4b (e.g. between clamping jaws). The substrate holders 4a, 4b can be coupled in the transverse direction via a gear unit 6a (e.g. including a leadscrew) driven by an actuator 6b, which ensures that the substrate holders 4a, 4b can be pulled apart precisely and parallel in the transverse direction (D1, D2), thereby creating a precisely defined tension on the conductor sections 100 (opposite forces F1, F2). At the same time, the constrictions 101 are heated by a suitable heater 5 so that the yield strength in the area of the respective constrictions 101 can be exceeded simultaneously and in a controlled manner for all conductor sections 100. This allows the generation of the ductile fractures 102 at the constrictions 101. The heater 5 can be configured to direct heated air 5a via a nozzle 5b onto the sections 100 of the conductors 10 in order to heat the sections 100.

FIG. 4 shows an embodiment of an implantable multielectrode array 1, wherein the substrate 200 has a curved shape. Particularly, the substrate 200 includes a first portion 201 integrally connected to a second portion 202 of the substrate 200, wherein the second portion 202 extends at an angle A (e.g. 90°) with respect to the first portion 201. Particularly, the second portion 202 can extend perpendicular to the first portion 201. Furthermore, particularly, the surface 200a of the substrate 200 from which the electrodes 3 of the implantable multielectrode array 1 protrude is formed by a face side of the second portion 202, so that particularly the electrodes 3 extend parallel to one another at the angle A with respect to the first portion 201 of the substrate 200. Such a configuration of the multielectrode array 1 is adapted to be implanted into the patient such that the first portion 201 of the substrate 200 can extend along the cortex C, wherein the second portion 202 of the substrate extends towards the cortex C, such that the tips 30 of the electrodes 3 can be inserted into the cortex C of the brain B of the patient. Particularly, the electrodes 3 of the array 1 shown in FIG. 4 can be constructed and include the dimensions according to the embodiments described herein.

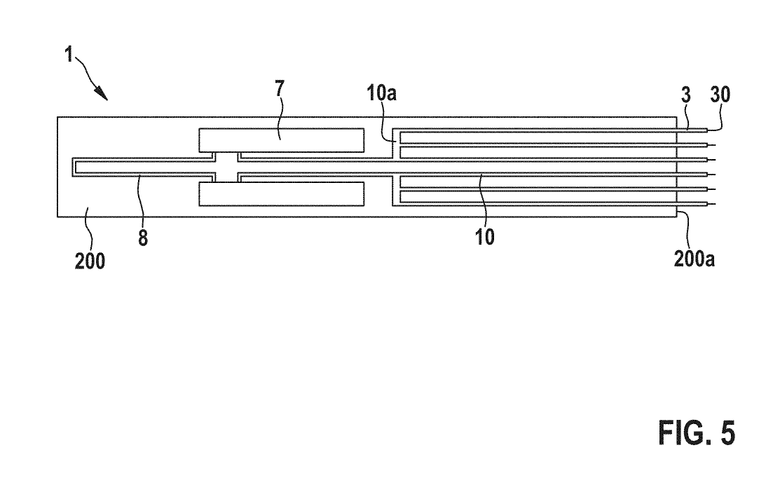

Furthermore, FIG. 5 shows, a diagrammatic illustration of an embodiment of an implantable multielectrode array 1, which includes a multiplexer chip 7 embedded into the substrate 200 for passing electrical signals to individual electrodes. Individual conductors 10 that end in electrodes 3 of the multielectrode array 1 can be connected by vertical connections 10a via which these conductors are then connected to the multiplexer chip 7.

Furthermore, the implantable multielectrode array 1 according to FIG. 5 can include an electrical coil for receiving data and electrical energy transmitted to the implantable multielectrode array 1, wherein also the coil 8 is preferably embedded into the substrate 200. Particularly, in detail, the electrodes 3 of the array 1 shown in FIG. 5 can be constructed and include the dimensions according to the embodiments described herein.

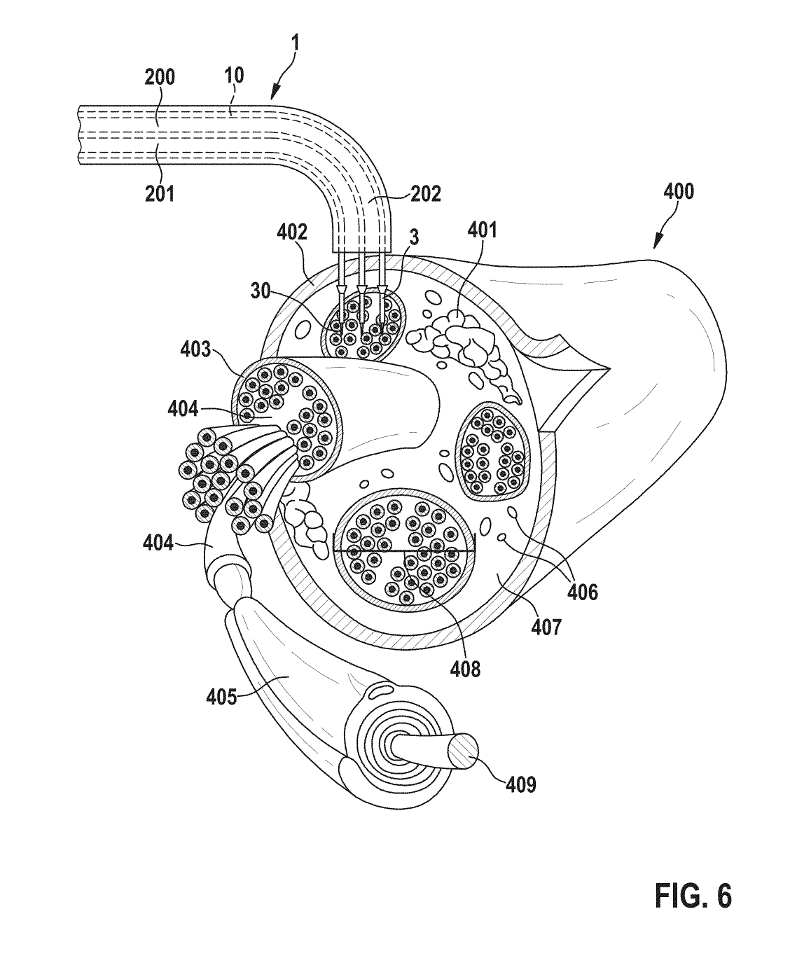

An application of the multielectrode array is shown in FIG. 6. Here, the multielectrode array 1 is applied to a peripheral nerve 400. Peripheral nerves are the part of the nervous system which is outside of the spinal cord. The peripheral nerve 400 includes epineurium 402, adipose tissue 401, blood vessels (artery and vein) 406, loose connective tissue 407, and fascicle 408. The fascicle includes perineurium 403, endoneurium 404, Schwann cell 405, and axon 409. Usual dimensions of the peripheral nerves are: collagen molecules: 1.3 nm, single nerve fiber: 2-5 μm, fascicle: 50-300 μm, and nerve fiber: 300-500 μm. The tensile modulus is approximately 0.5 MPa.

The dimension of the peripheral nerve depends on the number of independent nerve fibers which are combined into one bundle. For nerves which go into an arm or a leg that might be a large number, as every different muscle needs a couple of different nerve fibers. If selective stimulation of a single nerve fiber shall be accomplished, the corresponding electrode has to be thin and stiff to extend from the outside of the nerve bundle to the fascicle inside the nerve bundle.

The present disclosure describes how very thin and long, insulated needles (electrodes) disposed in a row configuration can be produced. With appropriate mechanical construction such rows of thin needles can be made such that they can easily be implanted and protrude from the outside of the nerve bundle into a fascicle as shown in FIG. 6. Here, each needle (electrode 3) contacts exactly one nerve fiber in the fascicle. The dimensions of the electrodes are: needle (electrode) diameter: 10-20 μm, tip (electrode tip 30) radius: 2-5 μm, and needle length: 50-150 μm.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

1. A method for producing an implantable multielectrode array, the method comprising the following steps:
a) providing a substrate, providing a plurality of conductors on the substrate, and providing each conductor with a section having a constriction, the sections extending parallel to one another in a first direction;
b) removing a portion of the substrate to form a first substrate part and a separate second substrate part being separated by a gap, each section extending in the first direction from the first substrate part across the gap to the second substrate part; and
c) exerting a first force on the first substrate part and exerting a second force on the second substrate part and heating the sections to generate a fracture of a respective section at a respective constriction, the fracture separating the respective section into an electrode protruding from the first substrate part and an electrode protruding from the second substrate part.

2. The method according to claim 1, wherein the respective fracture is a ductile fracture.

3. The method according to claim 1, which further comprises generating a plurality of first and second substrate parts by repeating steps a) to c).

4. The method according to claim 3, which further comprises forming the implantable multielectrode array by bonding together a plurality of substrate parts including at least one of the first or second substrate parts to form a substrate of the implantable multielectrode array having the electrodes protruding from a surface of the substrate formed by the plurality of bonded together substrate parts.

5. The method according to claim 1, wherein the substrate includes or is formed of a thermoplastic polymer.

6. The method according to claim 1, which further comprises forming a respective conductor from a photolithographically defined conductor track applied to the substrate by galvanic reinforcement of the respective conductor track.

7. The method according to claim 1, which further comprises an additional step of coating a tip of each electrode with a conductive coating.

8. A device for producing an implantable multielectrode array, the device comprising:
a first substrate holder for holding a first substrate part carrying a plurality of conductors having parallel sections with constrictions;
a second substrate holder for holding a second substrate part carrying a plurality of conductors having parallel sections with constrictions;
the sections of the conductors of the first substrate part being separated from the sections of the conductors of the second substrate part by a gap;
a heater for heating the sections of the conductors; and
an actuator configured to move the two substrate holders apart for exerting a first force on the first substrate part, for exerting a second force on the second substrate part and for generating a fracture of a respective section at a respective constriction, the fracture separating the respective section into an electrode protruding from the first substrate part and an electrode protruding from the second substrate part.

9. An implantable multielectrode array, comprising:
an insulating substrate having a surface;
a plurality of metallic conductors embedded in said insulating substrate;
each of said conductors having an end section protruding from said surface of said substrate; and
each of said end sections forming a respective electrode including a drawn tip.

10. The implantable multielectrode array according to claim 9, wherein each respective electrode includes a fracture surface of a ductile fracture at said drawn tip of said respective electrode.

11. The implantable multielectrode array according to claim 9, wherein said substrate includes or is formed of a thermoplastic polymer or a liquid crystal polymer.

12. The implantable multielectrode array according to claim 9, wherein each respective tip is coated with a conductive coating, or platinum, or iridium, or an alloy of platinum and iridium.

13. The implantable multielectrode array according to claim 9, wherein said drawn tip of each respective electrode protrudes past said surface of said substrate over a length lying in a range of from 0.02 mm to 3 mm.
